(19) United States
(12) Patent Application Publication
(10) Pub. No.: US 2022/0030882 A1
(43) Pub. Date: Feb. 3, 2022

(54) Composition and Method to Remove Parasites From Fish and to Prevent or Treat Infestation or Infection of Parasites on Fish

(71) Applicant: INAKVA AS, Skånevik (NO)

(72) Inventor: Elisabeth Winterthun, Skånevik (NO)

(21) Appl. No.: 17/290,528

(22) PCT Filed: Nov. 1, 2019
(86) PCT No.: PCT/NO2019/050238

§ 371 Date: Apr. 30, 2021

(30) Foreign Application Priority Data
Nov. 2, 2018 (NO) 20181404

(51) Int. Cl. A01N 65/38; A01K 61/13

(52) U.S. Cl. CPC A01N 65/38; A01K 61/13

(57) ABSTRACT

A composition and a method with active agents to inactivate or kill parasites that attack fish and to loosen parasites that are fastened to the fish are described. The composition can be used for the prevention of and treatment of infestations or infections from parasites on fish.

### BACKGROUND

The disclosed embodiments relate to a composition to prevent or treat infestations or infections of parasites on fish and a method to prevent parasites attacking and attaching to the fish and/or loosening parasites that are fastened to the fish and/or killing parasites.

The fish farming industry in Norway has become one of the most important industries and exporters in the country. For more than 40 years Norway has worked on improving the quality of food fish and is the world largest producer of salmon today. Norwegian salmon is the very trademark one associates with Norwegian fjords; pure water and high food quality. Norway exports salmon to more than 100 countries throughout the world. The industry is still under considerable pressure to produce more fish, at the same time as the production is vulnerable to infections and infestations from parasites.

One of the greatest challenges for the fish farming industry is salmon lice. The salmon louse (Lepeophtheirus salmonis) is a small parasitic crustacean with an ability to penetrate the slime layer of the salmon. It eats the salmon skin, slime and blood and create wounds in the fish. This exposes the salmon to other bacterial and viral infections that can lead to diseases and death.

Many different methods for treatment are in use or are under development to limit the danger of infection. Many of the known methods have unfortunate environmental consequences and are detrimental to the fish welfare. Several of the treatment methods used today have increasing problems with the development of resistance in the salmon lice. Many of the treatments focus only on individual or a few of the development stages of the salmon louse. Thus, there is a need for new treatment forms to replace and complement the present treatment methods.

The life cycle of a salmon louse has ten different stages where it changes shell between each stage. In the early stage (the nauplii stage) the louse lives freely in the water and can spread out over large areas. The salmon louse then grows and gets to the stage where it goes from living freely in water to fastening onto a host (copepodite 1 and 2). After this, the salmon louse starts to take nourishment from the fish during the four stages which are called Chalimus 1-4. Thereafter, the salmon louse goes over to the “moving stage” where it lives freely on the fish, this is called the pre-adult stage. The last stage is the adult. A grown, sexually mature female can produce up to 11 pairs of egg strings which each contains hundreds of eggs after fertilisation. The total life cycle of a salmon louse can be as short as 21 days at about 14 degrees Celsius.

Examples of known treatment methods against salmon lice are Thermolicer and Optilicer where hot water is used and Hydrolicer and Skamik where flushing and pressure are used. There are also a number of different bathing treatments that use fresh water, hydrogen peroxide, Salsoman vet (Azamethiphos), Alphamax (Deltametrin) and Betamax (Cypermetrin), and functional feed materials such as Slice vet (Emamectin benzoate) and Releeze vet (Diflubenzuron).

Thus, it would be advantageous to provide new active compositions to fight parasites on fish, such as salmon lice.

It would further be advantageous if such new active compositions can be used in treatment methods such as bathing treatments where the fish stays in a fluid with an active ingredient for a certain time, sufficient for inactivation or killing of the parasites.

It would additionally be advantageous if the active compositions can be administered to the parasites or the fish in a different way than with a bathing treatment, and thus provide feed materials that contain active ingredients which prevent parasites from settling on the fish and infecting the fish.

### SUMMARY

The disclosed embodiments relate to a composition to be used in the prevention or treatment of infestations or infection of parasites on fish, characterised in that the composition is comprised of one or more capsaicinoids or analogues or salt thereof and acetic acid.

In a preferred embodiment the composition comprises one or more capsaicinoids chosen from the group that contains capsaicin, dihydrocapsaicin, nordihydrocapsaicin, homocapsaicin, homodihydrocapsaicin and nonivamide.

In a preferred embodiment the composition is an aqueous composition and pH of the composition is below 5, more preferred below 4.5, more preferred below 4, more preferred below 3.5 and more preferred below 3 and most preferred about 2.8.

In one embodiment the composition is comprised of acetic acid and that the pH is below 3, preferably about 2.8.

In one embodiment said capsaicinoid is capsaicin.

In one embodiment said capsaicinoid is from the plant family Capsicum.

In one embodiment said Capsicum is a chili pepper, preferably Cayenne.

In one embodiment said capsaicinoid has a concentration in said composition of from 0.01-5%, more preferred 0.01-1%, more preferred 0.01-0.1%.

In one embodiment said capsaicinoid has a concentration in said composition of 10-100 grams Capsicum per litre fluid, more preferred 30-70 grams Capsicum per litre fluid and most preferred about 50 grams Capsicum per litre fluid.

In one embodiment said parasites are ectoparasites.

In one embodiment said parasite is a Caligidae.

In one embodiment said Caligidae is chosen from the group that consists of Pseudocaligus, Caligus and Lepeophteirus.

In one embodiment said Caligidae is the salmon lice Lepeophtheirus salmonis.

In one embodiment said fish is a salmonoid (Salmonidae).

In one embodiment said salmonoid is chosen from, among others, Atlantic salmon, silver salmon, trout and char.

In one embodiment the composition is used in a bathing fluid for bathing treatment of said fish.

In one embodiment the time the fish spends in the bathing solution can be regulated and the residence time is preferably from 1-3600 seconds, more preferred from 5-1800 seconds, more preferred from 5-180 seconds, more preferred from 10-120 seconds, more preferred from 10-60 seconds, more preferred from 10-30 seconds.

In a second a method to prevent parasites from attacking and attaching onto a fish and/or to loosen parasites that are fastened onto the fish and/or to kill parasites is provided, characterised in that the fish is taken into a water bath to which is added a composition that is comprised of one or more capsaicinoids or analogues or salts thereof and acetic acid.

In a preferred embodiment the composition is comprised of one or more capsaicinoids chosen from the group that contains, among others, capsaicin, dihydrocapsaicin, nordihydrocapsaicin, homocapsaicin, homodihydrocapsaicin and nonivamide.

In a preferred embodiment the composition is an aqueous composition and the pH of the composition is below 5, more preferred below 4.5, more preferred below 4, more preferred below 3.5 and more preferred below 3 and most preferred about 2.8.

In one embodiment the composition is comprised of acetic acid and a pH below 3, preferably about 2.8.

In one embodiment said capsaicinoid is a capsaicin.

In one embodiment said capsaicinoid is from the plant family Capsicum.

In one embodiment said Capsicum is a chili pepper, preferably Cayenne.

In one embodiment said capsaicinoid has a concentration in said composition of from 0.01-5%, more preferred 0.01-1%, more preferred 0.01-0.1%.

In one embodiment said composition contains capsaicinoids from 10-100 grams Capsicum per litre fluid, more preferred 30-70 grams Capsicum per litre fluid and most preferred about 50 grams Capsicum per litre fluid.

In one embodiment said parasites are ectoparasites.

In one embodiment said parasite is a Caligidae.

In one embodiment said Caligidae is the salmon lice, Lepeophteirus salmonis.

In one embodiment said fish is a salmonoid (Salmonidae).

In one embodiment said salmonoid is chosen from, among others, Atlantic salmon, silver salmon, trout and char.

In one embodiment the residence time of the fish in the water bath can be regulated and the residence time is preferably from 1-3600 seconds, more preferred from 5-1800 seconds, more preferred from 5-180 seconds, more preferred from 10-120 seconds, more preferred from 10-60 seconds, more preferred from 10-30 seconds.

### DETAILED DESCRIPTION

We have found that capsaicinoid effectively fights salmon lice and can therefore be used to inactivate parasites and to prevent the parasites from infestation and attaching to the fish, such as salmon.

We do not know the biological effect mechanisms, but it is assumed that receptors interacting with capsaicinoid compounds plays a part, and it is expected therefore that all parasites that have such receptors will be influenced.

The tests that are described below have been carried out with salmon lice as an example of a parasite, but the inventive concept is not limited to salmon lice as the method will also be effective against other parasites.

Furthermore, the firsts tests on fish will be carried out with salmon, but the inventive concept is not limited to salmon, as it is expected that all fish that are attacked by parasites that are sensitive for capsaicinoid will experience an effect from the method.

Furthermore, the firsts active agents against parasites have been tested in a so-called bathing treatment, i.e. that the parasites and the fish stay a given time in a watery environment to which the active agents are added.

However, it is expected that the active agent has a general effect on the parasites and other methods to administer an active agent to the parasites are therefore possible. We know of many other chemical active agents that can be indirectly administered to the parasites, for example, in that an active agent is administered to the host, for example, via the feed or by injection.

### Example 1

### Manufacture of the Composition Containing an Active Agent

The first introductory tests have been carried out directly on salmon lice in that an active agent is added to the fluid in which the lice are contained.

The active agent is a capsaicinoid and a fluid is presented containing such capsaicinoids. Capsaicinoids can be found in plants of the Capsicum family, such as Cayenne.

500 g of chili of the type Cayenne (Scoville scale 30 000-50 000) is washed in clean water to rinse off any bacteria or pollutants that come from the chili production. Other types of chili with another content of capsaicinoids, such as capsaicin can be used, but one must correct for the amount of capsaicinoids in the solution.

500 g Cayenne including the stalks are chopped into a soft mass by the use of mechanical equipment.

One litre of pure water is added to the chili mass. The mixture is heated to boiling and then removed from the heat. The procedure is necessary to release as much capsaicin as possible, and also to ensure conservation by killing the bacteria that were not removed in the first washing process.

Capsaicin is not soluble in water and one litre of 35% acetic acid is mixed into the hot chili mass and blended thoroughly. Other concentrations of acetic acid can, of course, be used. Finally, one adds a further 8 litres of water. The total amount of fluid is then 10 litres+the mass of the organic mixture.

The mixture shall then be kept cool in airtight kegs, optimally around four degrees for 10-14 days. During the 14 days the mixture is blended four times. After 14 days the organic chili mass is removed by filtration. The fluid is poured into clean kegs. The result is 10 litres of fluid which contains capsaicinoids in the solution denoted “treatment fluid”. The pH of the solution is 2.8 and the solution has a dark orange colour and a pungent smell. The solution is named the treatment fluid or Lepeoid in the examples and the tables given below.

The shelf-life of the fluid with capsaicin is very good as both capsaicin and acetic acid are natural conservation agents. By storing at between 2-4 degrees Celsius it is assumed that it lasts for several years.

The most common capsaicinoid is capsaicin which has the following formula;

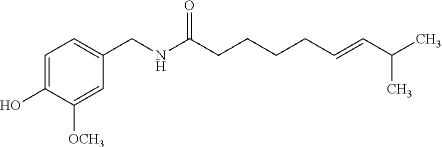 [SMILES: COc1cc(CNC(=O)CCCC/C=C/C(C)C)ccc1O]

Pure capsaicin is a colourless and odourless material with a waxy to crystalline consistency. It is very hydrophobic, but soluble in ethanol and other hydrophobic solvents. The melting point is 57-66 degrees Celsius.

Capsaicin is the molecule that can be found in chili plants and which causes the burning heat/pain one experiences when one consumes chili or get this on one's skin, in the eyes or mucosal membranes. Capsaicin binds to specific receptors, called the TRPV1 receptors which is a kind of pain receptor that registers heat. In humans, capsaicin will be able to trigger strong, but not very serious reactions when one comes into contact with a given concentration. For a long time, it was considered that fish did not have these receptors at all, but recent studies have shown that they can also be found in fish, but one is uncertain about the significance of this.

In chili, the strength of chili is determined according to the Scoville scale which is directly correlated to the amount of capsaicin in the chili fruit. The content of capsaicin can be determined with HPLC (High performance liquid chromatography).

Cayenne (which we have used in the development of the composition disclosed herein lies between 30 000-50 000 on the Scoville scale. Pure capsaicin powder has 16 000 000 which is the max value).

Capsaicin is as mentioned very hydrophobic, which means it does not dissolve easily in water. It is therefore dependent on a different solution fluid to become hydrophilic. If a person skilled in the art should find a different solvent than water it would be pertinent to use a different solvent such as, for example, ethanol, ether, benzene, methyl sulfoxide.

We have tested in experiments with lice the effect of ethanol as a solvent for the capsaicin. However, it turns out that a combination of capsaicin and ethanol has no effect on the mortality of salmon lice. To increase the solubility of capsaicin (by using ethanol instead of water or acetic acid) does not result in an increase in the effectivity of capsaicin on parasites.

Therefore, it is the specific combination of capsaicin and acetic acid which has a pronounced, positive effect on the lice. We have also tested other acids to see if it is the pH of the solution which provides the effect. If one uses citric acid one obtains the same pH (as with acetic acid) but the effect is much lower (data not show).

In addition, we have found that the combination of acetic acid and capsaicin provides a synergistic effect, and such synergistic effect is not something which a person skilled in the art would expect.

We have, as shown, used acetic acid. To use acetic acid as a solvent for Capsaicin is not an obvious solution for a person skilled in the art.

Capsaicin has an effect on parasites on its own and this effect is reinforced synergistically when one adds acetic acid to the mixture. We have tested the effect of i) acetic acid on its own and ii) capsaicin on its own and the sum of the effects on lice for each of these is considerably lower than for the combination of capsaicin and acetic acid.

Acetic acid can have several functions which we are investigating in tests. Acetic acid can have a conserving effect on the treatment fluid. Furthermore, acetic acid reduces the pH of the solution and the aqueous composition which results has a pH of 2.8. Acetic acid is also an irritant which can contribute to the effect on salmon lice.

### Example 2

Bathing Treatment of Parasites with a Fluid Comprised of Capsaicinoid and Acetic Acid

A series of tests has been carried out with salmon lice (Lepeophteirus salmonis) for the following stages of development: small movers, large movers and sexually mature salmon lice. The aim of the tests was to find out which concentration of capsaicinoid was appropriate to get a quick effect on salmon lice and their ability to suck on to the wall of the container at the same time before the salmon lice died.

Clean, white containers were filled with 0.5 litres of seawater (pH 8.9) at a temperature of 16.6 degrees Celsius. Five to eight live salmon lice at different stages of development where placed in each container.

Before pouring in the mixture as given in example 1 at different concentrations, the water in the tank was stirred gently so that the fluid should mix swiftly with the content in the container.

How much fluid with an active agent which was added to the seawater can be found in table 1 given below.

TABLE 1
Results for various concentrations of treatment
methods and lethality in salmon lice.

| | | | | Time to | |
|---|---|---|---|---|---|
| | | Temper- | Time to | death all | |
| Concentration | pH* | ature | reaction | stages | Comments |
| 10 ml treatment | 4.48 | 16.7 | Direct | Approx. 5 | Large movers |
| fluid per liter | | | | minutes | die after 3 min |
| sea water | | | | | |
| 15 ml treatment | 4.12 | 16.6 | Direct | Approx. 4 | |
| fluid per liter | | | | minutes | |
| sea water | | | | | |
| 20 ml treatment | 3.99 | 16.6 | Direct | 1 min and | |
| fluid per liter | | | | 20 sec | |
| sea water | | | | | |

*pH in treatment bath (mixture of fluid with active ingredients and sea water)

### Example 3

### Reaction Pattern and Lethality for Salmon Lice

In this experiment we considered fish welfare. Many studies have been carried out with salmon and its ability to tolerate pH changes but most of the studies are associated with low pH and increased aluminium levels over time. It is known that a pH down to 4.0 is acceptable to salmon over long periods, but at pH values below 4.0 the stress level for the salmon being tested increases. Therefore, we wanted to focus on concentration levels of the treatment fluid which are as effective as possible without affecting the welfare of the salmon.

TABLE 2
Result of reaction pattern and lethality in salmon lice
by addition of 20 ml treatment fluid per liter sea water

| | | Time to | Release |
|---|---|---|---|
| Trial number | pH | reaction | time* |
| 1 | 4.14 | Direct | 30 sec |
| 2 | 4.08 | Direct | 24 sec |
| 3 | 4.0 | Direct | 14 sec |
| 4 | 4.02 | Direct | 14 sec |
| 5 | 3.98 | Direct | 12 sec |
| 6 | 4.12 | Direct | 35 sec |
| 7 | 4.01 | Direct | 14 sec |
| 8 | 3.98 | Direct | 17 sec |
| 9 | 4.02 | Direct | 14 sec |
| 10 | 4.0 | Direct | 15 sec |

*Time it takes from addition of treatment fluid to the salmon lice starts to detach from the container wall.

In repeated experiments with 20 ml treatment fluid per litre seawater, we found that an average time from added treatment fluid to the salmon lice reacting to the situation is immediate. Jerks and contractions are observed in the salmon lice. After an average time of 18 seconds, the salmon lice attempt to flee from the ambient conditions. They let go of the container wall and try to swim away. A short time afterwards (seconds) they turn onto their back and fall towards the bottom of the container. They can make several attempts to swim away. Alternatively, they just loosen themselves from the container wall and sink to the bottom. The majority of the salmon lice were dead after 1 minute and 20 seconds.

In the experiments there was a trend that large sexually mature salmon lice died first, followed by the large movers and finally the small movers.

### Example 4

### The Effect of Increasing Concentration and Temperature on Copepodites

The tables 3-6 show the effect of increasing concentration and temperature on movement (time until movements stop). The tables show that increasing concentration and increasing temperature reduced the time for the movement of the parasites stop.

TABLE 3
Results of the response of copepodites (time to stop moving in seconds)
to Lepeoid concentrations (5, 10, 20 mL/L), tested the first time at 12° C.
Bioassays were run in triplicates (test 1-3) with ~20-30 copepodites.
12° C._1

| | | | Stop | |
|---|---|---|---|---|
| | | | moving | |
| Concentration | Test | Response | (s) | Notes |
| 20 mL/L | 1 | Direct (*) | 94 | 3 move after 60 s |
| | 2 | Direct (*) | 161 | 4 move after 90 s |
| | 3 | Direct (*) | 92 | 4 move after 60 s |
| 10 mL/L | 1 | Direct (**) | 96 | 3 move after 50 s |
| | 2 | Direct (**) | 114 | 2 move after 60 s |
| | 3 | Direct (**) | 137 | 2 move after 60 s |
| 5 mL/L | 1 | ca. 30 s | 209 | 6 move after 60 s |
| | 2 | ca. 30 s | 495 | 6 move after 60 s |
| | 3 | ca. 30 s | 459 | 8 move after 60 s |

Notes for tables 2 to 5
(*) Direct response, some show extreme high activity, some directly stop moving.
(**) Direct response, but less extreme than with 20 mL/L.

TABLE 4
Results of the response of copepodites (time to stop moving in seconds)
to Lepeoid-concentrations (5, 10, 20 mL/L), tested the second time
at 12° C. Bioassays were run in triplicates (test 1-3)
with ~20-30 copepodites.
12° C._2

| | | | Stop | |
|---|---|---|---|---|
| | | | moving | |
| Concentration | Test | Response | (s) | Notes |
| 20 mL/L | 1 | Direct (*) | 90 | 4 move after 40 s |
| | 2 | Direct (*) | 86 | 2 move after 30 s |
| | 3 | Direct (*) | 116 | 3 move after 30 s, |
| | | | | 1 quiver after 90 s |
| 10 mL/L | 1 | Direct (**) | 390 | 3 move after 110 s, |
| | | | | 1 after 195 s |
| | 2 | Direct (**) | 224 | 3 move after 90 s, |
| | | | | 1 after 150 s |
| | 3 | Direct (**) | 254 | 5 move after 60 s, |
| | | | | 1 after 190 s |
| 5 mL/L | 1 | ca. 10 s | 826 | 1 move after 9.5 min |
| | 2 | ca. 10 s | 1131 | 2 move after 15.5 min |
| | 3 | ca. 10 s | 1210 | 2 move after 18.5 min |

TABLE 5
Results of the response of copepodites (time to stop moving in seconds)
to Lepeoid concentrations (5, 10, 20 mL/L), tested at 20° C.
Bioassays were run in triplicates (test 1-3) with ~20-30 copepodites.
20° C.

| | | | Stop | |
|---|---|---|---|---|
| | | | moving | |
| Concentration | Test | Response | (s) | Notes |
| 20 mL/L | 1 | Direct (*) | 67 | 5 move after 20 s; |
| | | | | 1 after 50 s |
| | 2 | Direct (*) | 45 | 1 moves after 30 s |
| | 3 | Direct (*) | 51 | 2 move after 30 s |
| 10 mL/L | 1 | Direct (**) | 84 | 3 move after 50 s |
| | 2 | Direct (**) | 70 | 1 moves after 50 s |
| | 3 | Direct (**) | 67 | 1 moves after 55 s |
| 5 mL/L | 1 | ca. 10 s | 250 | 2 move after 60 s |
| | 2 | ca. 10 s | 248 | 2 move after 120 s |
| | 3 | Direct (**) | 295 | 5 move after 110 s |

TABLE 6
Results of the response of copepodites (time to stop moving in seconds)
to Lepeoid concentrations (5, 10, 20 mL/L), tested at 7° C. Bioassays
were run in triplicates (test 1-3) with ~20-30 copepodites.
7° C.

| | | | Stop | |
|---|---|---|---|---|
| | | | moving | |
| Concentration | Test | Response | (s) | Notes |
| 20 mL/L | 1 | Direct (*) | 130 | 2 move after 60 s; |
| | | | | ‘quivering’ after 90 s |
| | 2 | Direct (*) | 119 | 3 move after 60 s; |
| | | | | ‘quivering’ after 90 s |
| | 3 | Direct (*) | 115 | 3 move after 60 s; |
| | | | | ‘quivering’ after 86 s |
| 10 mL/L | 1 | Direct (**) | 234 | 3 move after 120 s |
| | 2 | Direct (**) | 214 | 2 move after 150 s |
| | 3 | Direct (**) | 194 | 3 move after 140 s |
| 5 mL/L | 1 | Direct (**) | 1275 | 2 move after 20 min. |
| | 2 | Direct (**) | 1200 | 3 move after 15.5 min |
| | 3 | Direct (**) | 1290 | 3 move after 15 min |

### Example 5—Effect of Increasing Concentration and Temperature on Adult Lice

When the active composition was introduced to adult lice the initial response was, in the main, that male lice spin round for a short period (seconds). The temperature and concentration influence the time until all movements have stopped (tables 7-9).

TABLE 7
Results of the response of adult lice and time to stop moving to Lepeoid-concentrations
(10, 20 mL/L), tested the first time at 12° C. Bioassays were run in triplicates (test 1-3)
with ~5 adult lice, except for the 20 mL/L, which was run 5 times.
12° C.

| | | | | Stop | |
|---|---|---|---|---|---|
| | | | | moving | |
| Concentration | Test | M/F | Response | (s) | Notes |
| 20 mL/L | 1 | 3/2 | Direct, but less extreme | 450 | 5 min. lice start to bend |
| | | | as with copepodites | | |
| | 2 | 2/3 | No ‘panicky’ responce | 342 | 4.75 min all bended |
| | 3 | 3/2 | | 226 | 1 min all move. 3 min. |
| | | | | | 1 male moves |
| | 4 | 4/1 | 1 directly moves, others | 440 | 4.3 min 1 male moves |
| | | | stop being active | | |
| | 5 | 4/1 | Direct | 490 | 90 s all move. 5.5 min |
| | | | | | 3 males move |
| 10 mL/L | 1 | 3/2 | 1 M and 1 F started to | 1350 | 6.5 min all move. |
| | | | swim directly | | |
| | 2 | 5/0 | | 1200 | 7.5 min all move |
| | 3 | 0/5 | | 1020 | 5 min. 4 stuck to the size |

Notes for this and following tables:
M/F = male/female numbers
w/prov = with provocation, slight tail pinch
Response was noted when different from general response of males (see text).

TABLE 8
Results of the response of adult lice and time to stop moving to Lepeoid-concentrations (10, 20 mL/L),
tested the first time at 20° C. Bioassays were run in triplicates (test 1-3) with ~5 adult lice.
20° C.

| | | | | Stop | |
|---|---|---|---|---|---|
| | | | | moving | |
| Concentration | Test | M/F | Response | (s) | Notes |
| 20 mL/L | 1 | 2/3 | | 425 | 50 s 1 F bends, 80 s all move w/prov; |
| | | | | | 200 s all bended |
| | 2 | 3/2 | Male repond directly | 430 | 80 s 1 F does not respond w/prov, |
| | | | for some seconds | | 110 s bending starts |
| | 3 | 1/4 | Male responds directly | 310 | 100 s 1 F moves, 4 min. Male move |
| | | | | | w/prov. |
| 10 mL/L | 1 | 2/3 | Direct response male | 750 | 60 s 2 M/1 F move & 2 F w/prov, |
| | | | | | 5.5 min. Bending start, move w/prov |
| | 2 | 4/2 | | 764 | 60 s all move w/prov, 9.5 min only 1 M |
| | | | | | moves w/prov |
| | 3 | 2/3 | indirect response | 630 | 60 s all move, 120 s males move, females |
| | | | | | w/prov, 9.5 min 1 F bends, 1 M w/prov |

TABLE 9
Results of the response of adult lice and time to stop moving to Lepeoid concentrations (10, 20 mL/L),
tested the first time at 7° C. Bioassays were run in triplicates (test 1-3) with ~5 adult lice.
7° C.

| | | | | Stop | |
|---|---|---|---|---|---|
| | | | | moving | |
| Concentration | Test | M/F | Response | (s) | Notes |
| 20 mL/L | 1 | 2/4 | Strong response all | 875 | 5 min all bended, 7.5 min move w/prov |
| | 2 | 1/4 | | 821 | 90 s all active w/prov, 9 min all bended |
| | 3 | 1/4 | | 920 | 100 s all move, 7 min 1 M and 1 F move a little |
| 10 mL/L | 1* | 2/4 | | 1800 | 120 s all move, 6 min. started to bend, |
| | | | | | 22 min. 2 F lifeless |
| | 2* | 4/1 | | 1800 | 7 min all move, after 30 min. 1 F moves |
| | 3* | 3/4 | | 1920 | 5 in. all move.; 23 min. 1 F stopped |
| | | | | | responding to trigger |

*Tests 1 and 2 were kept in Lepeiod solution, 3 was transferred to clean seawater, in the afternoon, no movement was observed in tests 1 and 2.

### Capsaicin

Not much can be found in the literature about capsaicin in a marine environment. Previous descriptions relate to capsaicin being used in bottom lubrication agents used on boats and other submersible surfaces. Capsaicin is described as a natural, not poisonous ingredient. It is a stable, organic alkaloid. The challenge is the poor water solubility of capsaicin. It is said that to use capsaicin as an active ingredient in bottom lubricants on ships leads to a relatively small risk for the marine environment. Capsaicin is biologically broken down on land. It is broken down by bacteria and one half will be broken down in two to eight days. There is little risk for leaks to the groundwater. It does not evaporate. A strong smell and taste means that animals stay away.

Capsaicin is not absorbed through the skin but the stomach. It is broken down in the liver. There is no information about the effect during pregnancy or on breastmilk. Capsaicin can influence resistance in air passages. Therefore, one can assume that people with obstructive lung diseases can be sensitive to capsaicin. Capsaicin can irritate skin and the mucosal membranes.

### Acetic Acid

Acidification of the ocean is a global problem. Emission of CO₂ contributes to 30% of the acidification. In the fjords the environment is dynamic and changes according to the temperature of the water and the currents. Acetic acid in large concentrations will be damaging to the marine environment. The treatment fluid will have a concentration of acetic acid of 3.5% which is lower than ordinary household vinegar. Furthermore, after treatment in the bathing fluid, the fish will be transferred to the net cage with seawater and the treatment fluid does not need to be discharged into the sea.

### Analogues

The biological effect-mechanisms of the active compounds disclosed herein are not known, but as indicated above it is possible that they work via a receptor family called vanilloide receptors, subtype 1 (TRPV 1). All capsaicinoid compounds that work via this receptor family are considered to be capable of inactivating or killing parasites.

In the preferred embodiments of the innovation, such capsaicinoid compounds will be manufactured from plants that contain such compounds, such as Cayenne pepper. In other embodiments the capsaicinoid compounds will be manufactured synthetically, or they can be manufactured using bacteriological processes. Furthermore, analogues of the capsaicinoid compounds are within the scope of this invention disclosure. Such analogues have the same base structure as capsaicinoid but have different substituents in non-essential places in the molecule.

The inventive embodiments also include different salt forms of the capsaicinoid compounds.

1-33. (canceled)

34. A method for deterring parasites from attacking and fastening to fish or for loosening parasites fastened to fish or killing parasites, comprising:
taking fish into a water batch;
adding a composition comprising (a) one or more capsaicinoids, or analogues or salts thereof, and (b) acetic acid to the water batch, thereby deterring parasites from attacking and fastening to fish, loosening parasites fastened to fish, and killing parasites present in the water batch.

35. The method according to claim 34, wherein one or more capsaicinoids is selected from the group consisting of capsaicin, dihydrocapsaicin, nordihydrocapsaicin, homocapsaicin, homodihydrocapsaicin and nonivamide.

36. The method according to claim 34, wherein the composition is an aqueous composition having a pH below 5.

37. The method according to claim 36, wherein the composition has a pH below 3.

38. The method according to claim 34, wherein said one or more capsaicinoid is capsaicin.

39. The method according to claim 38, wherein the capsaicin is provided from the plant family Capsicum.

40. The method according to claim 39, wherein the capsicum is a chili pepper.

41. The method according to claim 34, wherein said one or more capsaicinoid has a concentration in said composition within an approximate range of 0.01-5% by weight.

42. The method according to claim 34, wherein said one or more capsaicinoid is present in the composition in an amount within an approximate range of 10-100 grams per liter of fluid.

43. The method according to claim 34, wherein said parasite present in the water batch is an ectoparasite.

44. The method according to claim 43, wherein said parasite present in the water batch is a Caligidae.

45. The method according to claim 44, wherein said Caligidae is selected from the group consisting of Pseudocaligus, Caligus and Lepeophtheirus.

46. The method according to claim 34, wherein said parasite present in the water batch is salmon louse, Lepeophtheirus salmonis.

47. The method according to claim 34, wherein said fish is a salmonoid (Salmonidae).

48. The method according to claim 47, wherein said salmonoid is one or more from the group consisting of Atlantic salmon, silver salmon, trout and char.

49. The method according to claim 34, wherein a residence time of the fish in the water bath is regulated and said residence time is within an approximate range of 1-3600 seconds.

50. A composition for deterring or treating an infestation or infection of parasites on fish, comprising:
(a) one or more capsaicinoids, or analogues or salts thereof, and
(b) acetic acid.

51. Composition for use according to claim 50, characterised in that one or more capsaicinoids are chosen from the groups consisting of capsaicin, dihydrocapsaicin, nordihydrocapsaicin, homocapsaicin, homodihydrocapsaicin and nonivamide.

52. The composition according to claim 50, wherein the composition is an aqueous composition having a pH below 5.

53. The composition according to claim 50, wherein a concentration of capsaicinoids within the composition is within an approximate range of 0.01-5% by weight.
